(19) United States
(12) Patent Application Publication
(10) Pub. No.: US 2002/0087152 A1
(43) Pub. Date: Jul. 4, 2002

(54) Systems and methods for delivering a probe into tissue

(75) Inventors: Paul W. Mikus, Aliso Viejo, CA (US); Sanford Damasco, Irvine, CA (US); Douglas Owen Chinn, Arcadia, CA (US); Gregory Lee Kelly, Margarita, CA (US)

Correspondence Address: LYON & LYON LLP, 633 WEST FIFTH STREET, SUITE 4700, LOS ANGELES, CA, 90071, US

(73) Assignee: Endocare, Inc.

(21) Appl. No.: 09/755,956

(22) Filed: Jan. 4, 2001

(51) Int. Cl. A61B 18/02

(52) U.S. Cl. 606/21

(57) ABSTRACT

Apparatus and methods are provided for placing an introducer sheath into a subcutaneous tissue region, such as a prostate, e.g., to facilitate insertion of a cryogenic probe. In one embodiment, a dilation apparatus is provided that includes an introducer sheath containing a dilator that, in turn, contains a stylet. A pointed tip of the stylet is inserted into a patient's skin and the dilator is advanced over the inserted stylet to dilate surrounding tissue. The introducer sheath is then advanced over the dilator through the tissue to provide access to a target tissue region, such as the prostate. The stylet and dilator are removed from the introducer sheath, leaving the introducer sheath in place. A cryogenic probe may be inserted into the introducer sheath, and may be coupled to a cryosurgical system, e.g., to ablate or otherwise treat the target tissue region.

### FIELD OF THE INVENTION

This invention relates generally to the field of tissue ablation, more particularly to systems and methods for delivering a therapeutic device to an internal tissue region, and even more particularly to systems and methods for delivering a cryogenic probe and performing a cryogenic procedure with the probe to treat a target tissue region.

### BACKGROUND OF THE INVENTION

Cryotherapy is used to ablate tumors without gland removal, thereby avoiding the risks and discomfort associated with traditional surgical techniques, such as radical prostectomy. Rather than surgically removing diseased tissue, such as a tumor, one or more cryogenic probes may be percutaneously inserted into diseased tissue to ablate the tissue by freezing it until cell necrosis occurs. Although surgery is avoided, the diameter of a cryogenic probe (typically three millimeters (3 mm)) generally prevents the probe from being inserted directly into a patient, unlike a smaller instrument, such as a biopsy needle.

To introduce a cryogenic probe into a diseased prostate, a complicated procedure, such as that shown in FIGS. 17A-17E, has generally been used to dilate the tissue along an insertion path before the probe is finally introduced. As shown in FIG. 16, this procedure generally involves the use of a kit 3010 including five (5) or more components, namely a stylet 3012, a needle 3014, a guide wire 3018, one or more dilators 3022 (one shown), and an introducer sheath 3024. First, the hollow needle 3014 is disposed over the stylet 3012 such that a pointed tip 3016 of the stylet 3012 extends beyond a distal end 3017 of the needle 3014. As shown in FIG. 17A, the needle 3014 and stylet 3012 are inserted percutaneously through a patient's perineum 1050, either directly or through a small incision, and advanced through intervening tissue into a desired location 1052, e.g., within the prostate. The insertion may be monitored and/or guided using an imaging system, such as a transrectal ultrasound device.

As shown in FIG. 17B, once advanced to the desired location, the stylet 3012 is removed from the needle 3014, and a guide wire 3018 is directed into a lumen of the needle 3014 until a distal tip 3020 of the guide wire 3018, e.g., a “J” tip, is coextensive with or extends beyond the distal end 3017 of the needle 3014. With the guide wire 3018 manually held stationary, the needle 3014 is withdrawn from the patient. As shown in FIG. 17C, one or more dilators 3022 are then advanced over the guide wire 3018 to dilate the tissue surrounding the guide wire 3018, thereby creating an insertion path from the patient's skin to the desired location. As shown in FIG. 17D, an introducer sheath 3024 is then advanced over the dilator(s) 3022 such that a distal end 3026 of the introducer sheath 3024 is positioned at the desired location within the prostate. Finally, with the introducer sheath 3024 held in position, the dilator(s) 3022 is(are) removed, as shown in FIG. 17E.

A cryogenic probe (not shown) may then be inserted through the introducer sheath into the prostate. Optionally, this procedure may be repeated to introduce a plurality of probes into the prostate in a predetermined arrangement. The probe(s) may then be used to treat the prostate, e.g., to ablate tissue surrounding the probe(s).

The above-described procedure, while ultimately providing a dilated insertion path maintained by the introducer sheath that allows a cryogenic probe to be introduced, suffers from a number of disadvantages. First, because of the several steps involved, the procedure is time consuming. In addition, because the procedure involves advancing and withdrawing multiple instruments, there is a risk that the sheath, and consequently the probe, may be misplaced within the prostate. For example, it may difficult to maintain the guide wire completely stationary during removal of the needle or advancement of a dilator over it, resulting in the guide wire possibly moving away from the desired location. Further, inserting and withdrawing multiple instruments increases patient discomfort and increases the risk of infection.

Accordingly, a system that provides more efficient creation of a dilated insertion path, and/or that facilitates placement of an introducer sheath or cryogenic probe into an internal tissue region would be useful.

### SUMMARY OF THE INVENTION

The present invention is directed to apparatus, systems, and methods for delivering a therapeutic device to an internal tissue region, and more particularly to systems and methods for delivering a cryogenic probe and performing a cryogenic procedure with the probe to treat a target tissue region within a patient.

In accordance with one aspect of the present invention, an apparatus provides percutaneous access for performing a cryogenic procedure within a target tissue region of a patient. The apparatus includes a stylet having a proximal end and having a distal end for penetrating tissue. A dilator is slidably disposed over the stylet, the dilator having a proximal end and a tapered distal end. The dilator has a length that is less than a length of the stylet. An introducer sheath is slidably disposed over the dilator.

The stylet and/or dilator includes a mechanism on their proximal end(s) for preventing proximal movement of the dilator and/or sheath proximally from the stylet. For example, a lock assembly may be provided on the proximal ends of the stylet and the dilator for substantially securing the dilator axially with respect to the stylet such that the distal end of the stylet extends beyond the distal end of the dilator. Preferably, the lock assembly, e.g., including a luer lock, is configured for detachably securing the proximal ends of the stylet and the dilator with respect to one another.

Preferably, the stylet is removable proximally from within the dilator, and the dilator is movable proximally with respect to the introducer sheath for removing the dilator from within the introducer sheath. A cryogenic probe is insertable into the introducer sheath once the dilator is removed from within the introducer sheath.

In accordance with another aspect of the present invention, a method is provided for using the apparatus to provide percutaneous access to an internal tissue region, e.g., to perform a cryogenic ablation procedure. The method includes providing a stylet having a dilator fixed about a proximal portion of the stylet such that a distal portion of the stylet extends beyond a distal end of the dilator. An introducer sheath is disposed about the dilator that is preferably secured from proximal movement from the dilator. The distal portion of the stylet is inserted into the patient to form an insertion path. The dilator is released from the proximal portion of the stylet and advanced along the stylet to dilate the insertion path. Having dilated the insertion path, the introducer sheath may be advanced over the dilator and into the insertion path. With the introducer sheath in position in the insertion path, the stylet and dilator may be removed from the patient.

A cryogenic probe may then be advanced through the introducer sheath and into the target tissue region. Optionally, the sheath may then be withdrawn, leaving the cryogenic probe in place. Otherwise, the sheath may be left in place, e.g., to thermally insulate tissue along the insertion path from the probe. Additional probes may be inserted into adjacent tissue regions, e.g., in a predetermined relationship to the first probe, using a similar procedure. The probe(s) may be connected to a cryogenic cooling system, a control system, and/or an imaging system, e.g., to ablate or otherwise treat the target tissue region. In a preferred embodiment, the probe(s) may be used to ablate all or a portion of a prostate, e.g., to necrose a cancerous or benign tumor therein.

Other aspects and advantages of the present invention are disclosed by the following description and figures.

### DESCRIPTION OF THE DRAWINGS

The various aspects and features of the present invention may be better understood by examining the following figures:

FIG. 1A is a side view of a first embodiment of an apparatus for providing percutaneous access to an internal tissue region, in accordance with the present invention.

FIG. 1B is an exploded side view of the apparatus of FIG. 1A.

FIG. 2 illustrates the initial insertion of a stylet, according to one embodiment of the invention.

FIG. 3 illustrates the advancement of a dilator over the inserted stylet of FIG. 2.

FIG. 4 illustrates the advancement of an introducer sheath over the dilator of FIG. 3.

FIG. 5 shows the introducer sheath of FIG. 4 after removal of the stylet and dilator.

FIG. 6 is a side view of another embodiment of an apparatus for providing percutaneous access, including a plurality of nested dilators, in accordance with the present invention.

FIG. 7 is a schematic diagram of a cryosurgical system for use with an access apparatus, in accordance with the present invention.

FIG. 8 is a cross-sectional view of a portion of a human body showing portions of the cryosurgical system in place.

FIG. 9 is a lateral view of a portion of a human body showing positions of the cryosurgical system in place.

FIG. 10 is a cross-sectional view taken along lines IV-IV of FIG. 8.

FIG. 11 is a longitudinal cross-sectional view of one embodiment of a temperature probe for use with the cryosurgical system.

FIG. 12 is a longitudinal cross-sectional view of another embodiment of a temperature probe for use with the cryosurgical system.

FIG. 13 is a perspective view of a stereotactic guide for use with the cryosurgical system.

FIG. 14 is an elevational view of the stereotactic guide of FIG. 13.

FIG. 15 shows a visual display that may be used with the cryosurgical system.

FIG. 16 is a side view of a conventional kit used to insert an introducer sheath into a subcutaneous tissue region.

FIGS. 17A-17E show a conventional method for inserting an introducer sheath using the kit of FIG. 16.

### DETAILED DESCRIPTION

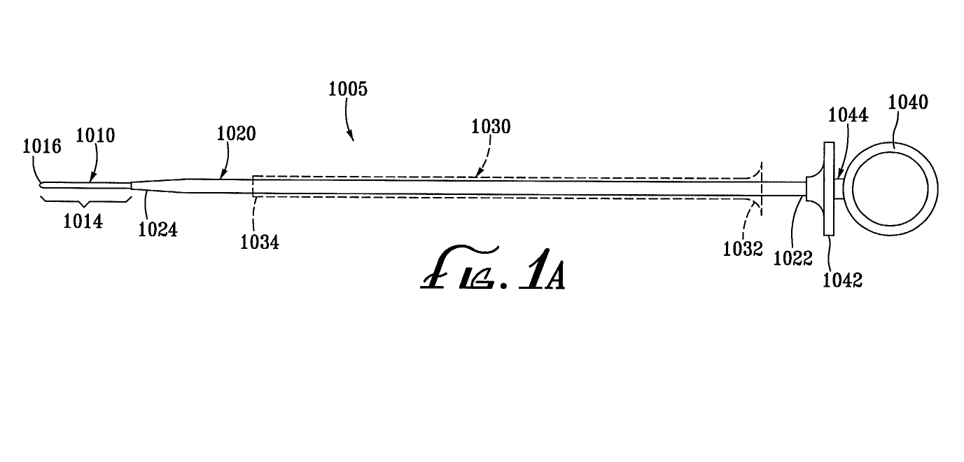

Turning now to FIGS. 1A and 1B, an apparatus 1005 for providing percutaneous access into a patient is shown that generally includes a stylet 1010, a dilator 1020, and an introducer sheath 1030 (shown in phantom in FIG. 1A), in accordance with the present invention. The stylet 1010 is a substantially rigid body including a proximal end 1012, and a distal portion 1014 terminating in a sharpened distal tip 1016. The stylet 1010 preferably has a substantially uniform outer diameter, e.g., 18 gauge or smaller. The distal tip 1016 is preferably configured for penetrating tissue, e.g., to puncture a patient's skin and/or to be introduced through tissue. The stylet 1010 may be formed from stainless steel or other biocompatible material.

The dilator 1020 is a tubular body including a proximal end 1022 and a tapered distal end 1024. The dilator 1020 may be formed from stainless steel, plastic, or other biocompatible material, and may be coated with teflon or other similar material to facilitate substantially atraumatic advancement through tissue. As shown in FIG. 1A, the dilator 1020 is slidably disposed over the stylet 1010, i.e., such that the stylet 1010 extends through a lumen 1026 in the dilator 1020. Preferably, the dilator 1020 has a length that is substantially less than a length of the stylet 101, such that the distal portion 1014 of the stylet 1010, e.g., at least about one (1) inch (25 mm) and preferably between about two and three (2-3) inches (25-38 mm), extends beyond the distal end 1024 of the dilator 1020. The dilator 1020 preferably has an inner diameter that corresponds substantially to the outer diameter of the stylet 1010, such that the tapered distal end 1024 of the dilator slides but remains in close proximity to the stylet 1010.

The introducer sheath 1030 is a substantially rigid or semi-rigid tubular body, e.g., made from plastic, such as polytetrafluorethylene, or other biocompatible material. The introducer sheath 1030 includes a proximal end 1032 and a distal end 1034, the distal end 1034 preferably being tapered. The introducer sheath 1030 preferably has a length that is substantially shorter than the length of the dilator 1020. The dilator 1020 is disposed within a lumen 1036 in the introducer sheath 1030 such that the distal end 1024 of the dilator extends beyond the distal end 1034 of the introducer sheath 1030 (shown in phantom). Preferably, the introducer sheath 1030 has an inner diameter that corresponds substantially to an outer diameter of the dilator, such that the tapered distal end 1034 tapers substantially to the outer diameter of the dilator 1020 while allowing the introducer sheath 1030 and dilator 1020 to slide with respect to one another.

To facilitate control of the apparatus 1005 during insertion into a patient, the stylet 1010 and/or dilator 1020 may include a mechanism for limiting movement of the stylet 1010, the dilator 1020 and/or the introducer sheath 1030 with respect to one another. For example, the stylet 1010 may include a thumb ring 1040 or other gripping member (not shown) on its proximal end 1012 that may prevent the dilator 1020 from sliding proximally off of the stylet 1010, e.g., as the stylet 1010 is being inserted into a patient. Preferably, the proximal end 1024 of the dilator 1020 includes a flange 1042 for preventing the sheath 1030 from sliding proximally off of the dilator 1020. The flange 1042 may have a curved distal side, e.g., to provide a finger grip that may further facilitate manipulation of the apparatus 1005 during insertion of the stylet 1010.

In addition, to substantially secure the stylet 1010 and dilator 1020 with respect to one another, a lock assembly, e.g., a conventional luer lock 1044, may be provided on the stylet 1010 and dilator 1020. For example, a male portion 1044b of the luer lock 1044 may be located on the proximal end 1010 of the stylet 1010 and a female portion 1044a located within the flange 1042 on the proximal end 1022 of the dilator 1020. Thus, the dilator 1020 and stylet 1010 may simply be twisted with respect to one another to lock or release the luer lock 1044.

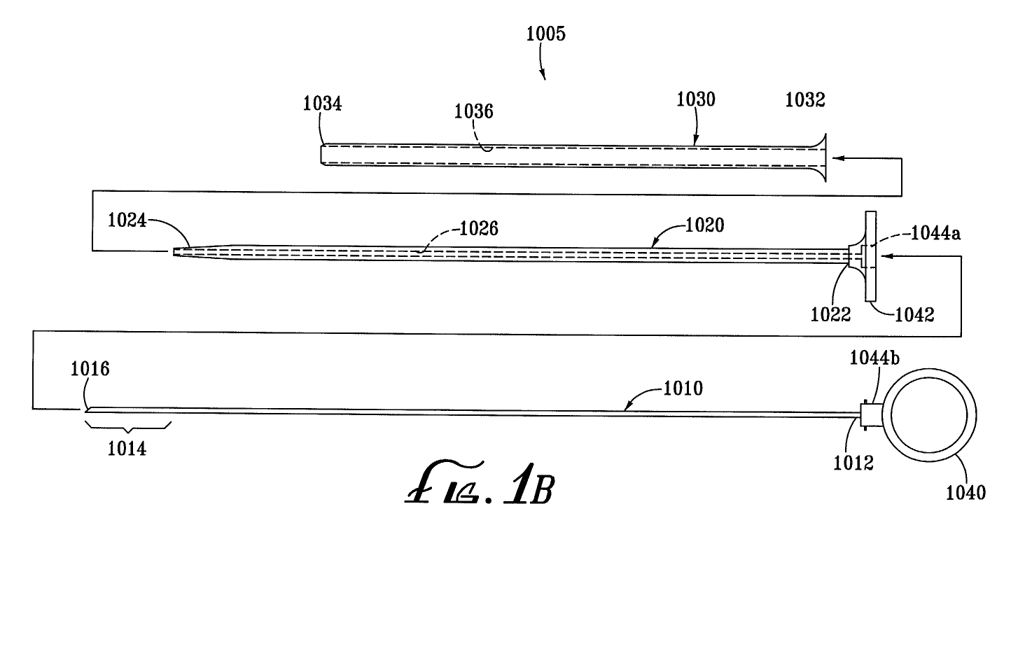

Turning now to FIGS. 2-5, a method is shown for placing the introducer sheath 1030 in a patient to provide percutaneous access to an internal tissue region, such as the prostate. All or a portion of the procedure may be imaged, e.g., to monitor locating the stylet 1010 within the patient, advancing the dilator 1020, and/or advancing the introducer sheath 1030, as described further below. The imaging methods may include, for example, ultrasound imaging, magnetic resonance imaging (“MRI”), or other methods known in the art.

First, the apparatus 1005 is provided substantially as shown in FIG. 1A. Then, as shown in FIG. 2, the distal tip 1016 of the stylet 1010 is inserted through the patient's skin 1050, e.g., through an incision (not shown) or directly into the patient' skin 1050. Because the stylet 1010 is analogous to a biopsy needle, it may easily be inserted deeply into the region of interest.

To aid in its insertion, the stylet 1010 is preferably inserted into a target tissue region without substantial hindrance from the dilator 1020. Thus, as described above, the dilator 1020 is preferably substantially shorter than the stylet 1010. When the proximal ends 1012, 1022 of the stylet 1010 and dilator 1020 are aligned, e.g., when the lock assembly 1044 is engaged, the distal portion 1014 of the stylet 1010 extends from the distal end 1024 of the dilator 1020.

Before inserting the stylet 1010, the luer lock 1044 may be twisted to ensure that the dilator 1020 is substantially secured and the distal portion 1014 of the stylet 10 is maintained beyond the distal end 1024 of the dilator 1020 during insertion. Alternatively, the mechanism on the stylet 1010 and/or dilator 1020 may include a pistol grip (not shown), e.g., on the flange 1042 and/or thumb ring 1040. Such a grip may facilitate applying considerable force as may be required to pierce fibroid tissue regions of the patient's body, e.g., as may be encountered after radiation therapy.

Turning to FIG. 3, having advanced the distal portion 1014 of the stylet 1010 into a target tissue region 1052, the dilator 1020 may be used to dilate an insertion path 1054 formed by the stylet 1010. In this stage of the procedure, the lock assembly 1044 on the stylet 1010 and dilator 1020 may be disengaged so that the dilator 1020 may be advanced distally over the distal portion 1014 of the stylet 1010. The substantially tapered distal end 1024 of the dilator 1020 substantially a traumatically dilates surrounding tissue, thereby expanding the diameter of the insertion path 1054. Thus, the insertion path formed initially by the insertion of the stylet 1010 may be dilated to conform to the outer diameter of the dilator 1020.

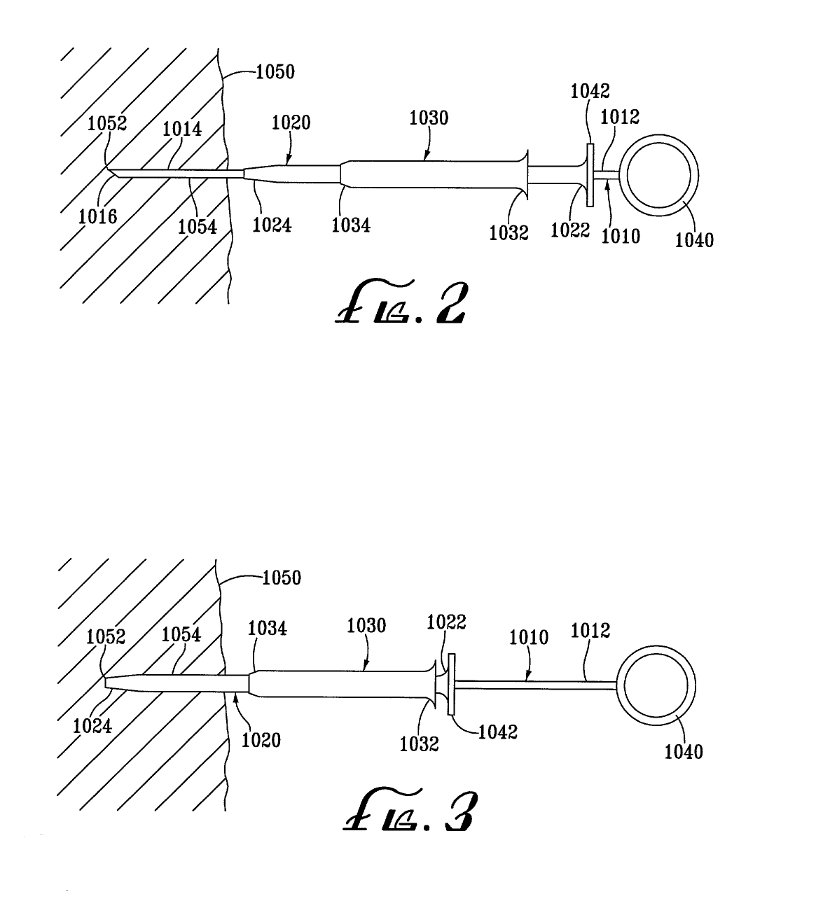

Turning to FIG. 4, the introducer sheath 1030 may then be advanced distally over the dilator 1020 into the dilated insertion path 1054. The advancement of the introducer sheath 1030 may be aided by the tapered distal end 1034 to the introducer sheath 1030, similar to the tapered distal end 1024 of the dilator 20.

As shown in FIG. 5, the dilator 1020 and stylet 1010 may then be withdrawn proximally from the introducer sheath 1030. An instrument, such as a cryogenic probe or other ablation device (not shown) may be inserted into the introducer sheath 1030 to provide further therapy, e.g., to ablate tissue in and/or around the target tissue region 1050. For example, a distal portion of a cryogenic probe may be inserted through the introducer sheath 1030 until it extends beyond the distal end 1034 of the introducer sheath 1030. The introducer sheath 1050 may be left in place, e.g., to insulate surrounding tissue proximal to the distal portion of the probe. Alternatively, after the probe is inserted into the target tissue region 1050, the introducer sheath 1030 may be removed. In addition, the apparatus 1005 or multiple apparatus (not shown) may be used to insert a plurality of probes into a target tissue region, e.g., in a predetermined arrangement with one another.

Turning to FIGS. 7-15, a cryosurgical system may be provided for treating a defined internal area of a living body using a plurality of cryogenic probes 8 that may be introduced using the apparatus 1005 described above. Alternatively, other instruments may be inserted into subcutaneous tissue regions of a patient using the apparatus 1005, such as other thermal probes, which may cool or heat the tissue, RF energy probes, radiation treatment devices, and the like.

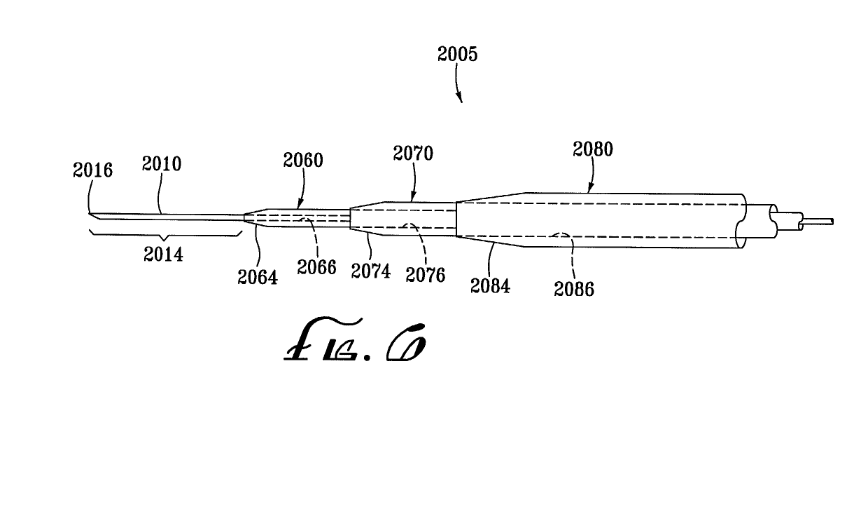

FIGS. 8 and 9 show the placement of portions of the cryosurgical system, particularly cryogenic probes 8, which may be inserted, in a defined region of a human body, specifically in a prostate gland. Alternatively, the system may be used to cryogenically treat a variety of internal tissue regions of a body, such as the liver, breast, eyeball, brain tissue and/or tonsils.

The cryosurgical system may include various major components, such as the discrete components described below. For example, as shown in FIG. 7, the system generally includes a cryogenic cooling system 2, such as that available from Endocare, Inc. located in Irvine, Calif. or Cryomedical Sciences Inc., located in Rockville, Md. The cooling system 2 controls circulation of coolant, such as argon or liquid nitrogen, from a reservoir 4 through a control system 6 to the cryogenic probes 8. Alternatively or additionally, the cooling system 2 may allow circulation of other fluids, such as Helium, which may be used to warm or otherwise provide additional temperature control of the probes 8. In a preferred embodiment, the cryogenic probes have a diameter of not more than about three millimeters (3 mm), and may be made of stainless steel. While only two probes are shown in FIG. 7, the cooling system 2 may control flow to any number of probes, e.g., five and preferably eight probes.

The cryosurgical system also may include an ultrasonic system 12 such as that made by Aloka Ultrasound of Wallingsford, Conn. The ultrasonic system generally includes an ultrasonic signal generator and processor 14 that is connected to an ultrasonic probe 16 to cause it to generate ultrasonic vibrations at window 18 and to receive those vibrations at window 20. These received vibrations may be converted to electrical vibrations that are supplied to processor 14 over connector 22. Connected to processor 14 is a display device 24, such as a CRT monitor, and a recording device 26, such as a video cassette recorder.

The cryogenic system further may include an integrated data processor 30 coupled to a temperature data acquisition system 32 via line 33. The temperature data acquisition system may electrically isolate the patient from the rest of the electronics. The data acquisition system, in turn, may be coupled to temperature probes 36, e.g., by lines 34. Although only two temperature probes are shown, any number of probes may be used, as will be appreciated by those skilled in the art. The data processor 30 may also be electrically coupled to the ultrasonic signal processor 14 through line 38 for receiving ultrasonic image information. A display device 40, such as a CRT monitor, may be connected to the data processor 30 to display an image that preferably incorporates both the ultrasonic image and temperature data, as described below. A recorder 42, such as a VCR, may also be connected to the data processor 30 for receiving and recording the visual information displayed on monitor 40 for future reference.

Finally, the cryosurgical system may include one or more temperature probes 50 that are connected to one or more hand held, battery powered meters 52 (such as a meter offered by Omega Engineering Inc. of Stanford, Conn.) via one or more lines 54. The temperature probes 50 may ensure that, should operational problems be encountered with either the ultrasonic system 12 or the temperature data acquisition system 32, or with data processor 30 or monitor 40, the system may be monitor temperatures sufficiently to allow cryogenic treatment to be discontinued in a controlled fashion, e.g., manually or automatically. Conveniently, these meters 52 may be mounted on or received in an appropriate holder (not shown) that supports them on or adjacent to monitor 40, e.g., to be in the same visual field as the monitor's screen, thereby facilitating both to be easily monitored. Additional information on a cryosurgical system appropriate for use with the present invention may be found in U.S. Pat. No. 5,647,868 issued to Chinn, the disclosure of which is expressly incorporated herein by reference.

Alternatively, the system may include a central console (not shown), including an internal computer or other processor. In addition, the console may include a cryogenic cooling system, connectors for receiving a data signal from an ultrasonic imaging apparatus, an integrated data processor, and/or a temperature data acquisition system, similar to the discrete components described above. The console may also include a display or other graphical output device, e.g., to display ultrasound images, temperature data, and the like, either separately or in conjunction with one another. The console may also include storage devices, such as disk drives and the like, for storing data for later use. The various probes, e.g., cryogenic probes and temperature probes, may be coupled to the console such that the console may control and/or monitor their use.

As previously indicated, FIGS. 8 and 9 illustrate use of the system to treat a prostate gland. FIG. 8 is a vertical cross-section through the medial plane of the lower torso region of a human male, while FIG. 9 is a lateral view through that region. In general, these views illustrate a prostate gland 60 surrounding a urethral tube 62 extending from bladder 64 to the tip of penis 66. Also shown is anus 70 and rectum 72. The cryogenic probes 8 may be percutaneously placed into target locations within the prostate gland 60, e.g., using the apparatus described above, possibly in conjunction with a stereotactic guide, such as that described below. Also, temperature probes 36 may be placed in or adjacent to the prostate gland, optionally using a stereotactic guide, such as the guide used for the cryogenic probes 8.

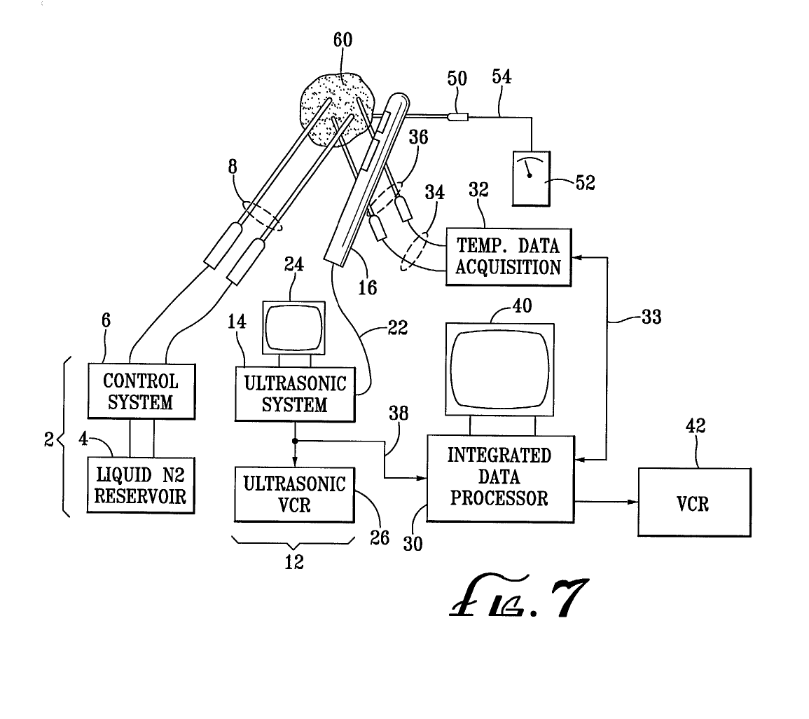

For a cryogenic surgical procedure, the preferred placement of the cryogenic probes and temperature probes is shown in FIG. 10, which is a cross-section taken on plane IV--IV of FIG. 8. The cryogenic probes 8 may be placed in the prostate gland 60 about the urethral tube 62 equally spaced about a target circle approximately half way between that tube 62 and the outer surface of the gland 60. Preferably, they are located to apply cryogenic cooling to the mid-portion of the gland 60, although, if the gland 60 is enlarged, it may be necessary to first place the probes 8 closer to the bladder, then later during the treatment to move them away from the bladder to treat the remaining apex portion of the gland 60. The temperature probes 36 may be placed in and about the gland 60, for example, as shown, with three temperature probes preferably being placed in the critical region between the prostate gland 60 and the adjacent wall of the rectum 72. The ultrasonic probe 16 may be placed through the anus and oriented such that its windows lie along the wall of the rectum 72 and face the prostate gland, as shown in FIGS. 8 and 9.

In general, during cryosurgical treatment of a target internal tissue region of a living body, the cryogenic probes 8 may be employed to cool the target tissue region to a fatal temperature, e.g., between about −15° C. and −44° C. It may be important to monitor the temperature of the region surrounding the target tissue region to ensure that the target temperature (usually a fatal temperature) is attained. As cooling occurs, an ice ball may form in the area being cooled. To prevent this cooling from extending to other regions of the body, it may be important to monitor temperatures accurately. In particular, when treating a prostate gland, it may be important to monitor the temperature between the gland and the wall of the rectum to ensure that the wall of the rectum is not cooled to a point where its viability is challenged, for this may result in fistulae in the rectum wall. For this reason, one of temperature probes 36′ may be placed posterior to the gland and anterior to the wall of the rectum. It may also be important to monitor growth of the ice ball as it forms in the prostate gland during cryogenic treatment to make sure that it extends from the base of the gland adjacent bladder 64 to its apex. Preferably, temperature probe 36″ is placed for this purpose.

An exemplary embodiment of a temperature probe 36 is shown in FIG. 11. The probe may include a length of hypodermic tubing 82, e.g., between about eight and nine (8-9) inches long, and having a diameter on the order of 0.035 inches (i.e. 18 gauge tubing), e.g., made of Type 316 stainless steel. The tube may be made by placing the necessary elements in a tube, then stretching the tube and included elements down to a desired outer diameter. Since this may result in a flexible tube, if such a construction technique is used, the resulting tube may be placed in another tube of appropriate rigidity. At its distal end 84, the tube may be closed, such as by a small amount of Type 316 stainless steel solder, and/or sharpened for percutaneous penetration into a defined area of a living body. The proximal end 86 of the tubing is received in a hollow cylindrical canister 88, also preferably made of Type 316 stainless steel. This canister may be on the order of ¼ inch in diameter and 2½ inches long. The proximal end of the tubing 82 preferably is attached to the end of canister 88 by a slug of cadmium free silver solder 90. A T-type thermocouple 92 may be positioned in the distal end of tubing 82 and may be connected by wires 94 to cable 34 within the canister. After this connection has been made, and the thermocouple tested, the canister may be filled with an epoxy 98 or other suitable material. Cable 34 extends to an appropriate plug for connection to the temperature data acquisition system 32. By this construction, the temperature probe may be cold sterilized using Cidex without degradation and/or gas sterilized by ethylene oxide [Eto].

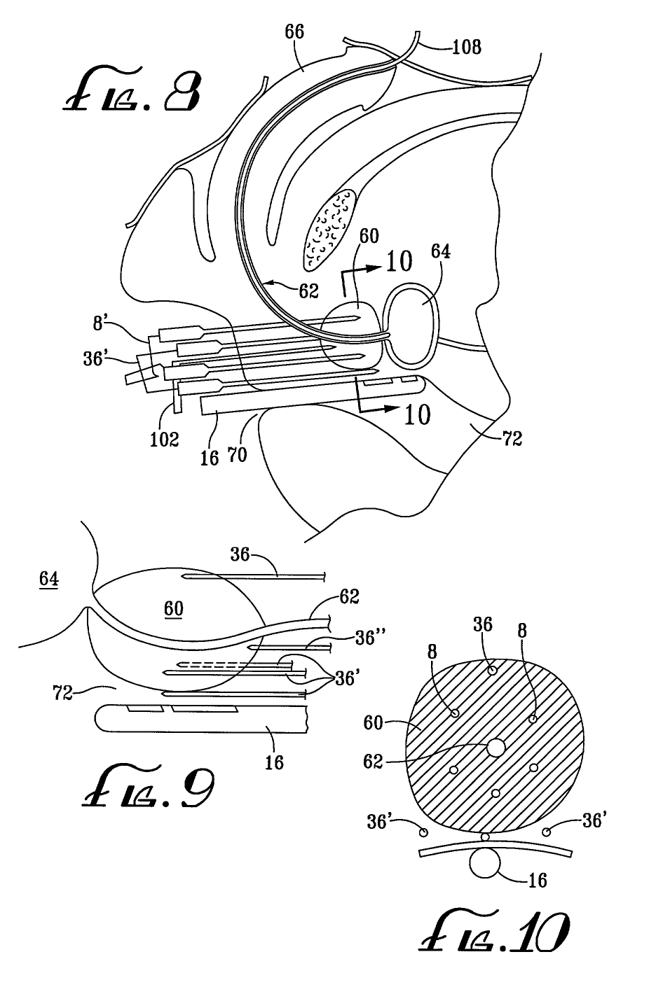

The probe shown in FIG. 11 may be appropriate for placement laterally of the prostate gland, e.g., as shown in FIGS. 8-10. It may also be desirable to have a probe designed for placement at the apex of the gland, such as probe 36″ shown in FIG. 9. As shown in FIG. 12, the construction of the probe 36″ may be substantially identical to the probe shown in FIG. 11, except that end 84′ may terminate in a point, an outer diameter of the tubing may be between about 0.040 or 0.065 inch, and the length of tubing 82′ may be on the order of about five to six (5-6) inches.

Placement of the temperature probe within a living body may be achieved in various ways, many of which are conventional to cryosurgical procedures. For example, using the ultrasonic probe, an 18 gauge Onik needle with a stylet (not shown) may be located and directed free hand or by an appropriate stereotactic guide, such as the guide described below. The needle may be appropriately directed to a desired location within or adjacent to the gland, after which the stylet may be withdrawn and a temperature probe inserted through the needle to the desired location. In general, the trocar or Onik needle (used to placed the cryogenic probes) may include a hollow tube whose inner diameter is slightly larger than the outer diameter of tubing 82. Normally, the trocar may be inserted percutaneously in the body, and then the probe may be passed through the trocar to cause the distal end 92 to project slightly beyond the end of the trocar, specifically a distance to unshield the thermocouple 92. In a preferred construction, the thermocouple is approximately one to two (1-2) millimeters long. To ensure that the thermocouple is unshielded, and to aid in ultrasonic visualizations of the probe, at least the distal portion of tubing 82 may include an echogenic coating 104 to enhance its ultrasonic image, and may be marked by break 106 to identify the distal portion of tubing 82 that houses the thermocouple 84. The base portion 86 of the probe may be marked with graduations, and these graduations may be employed to monitor, e.g., ultrasonically, when the tip of the probe begins to extend beyond the trocar. This may ensure that a sufficient length of the probe's tip extends past the tip of the trocar.

When cryosurgically treating a prostate gland, it is often desirable to circulate a warming fluid through the urethral tube. To this end, a catheter 108 may be inserted through the penis and employed to circulate water warmed to about forty four (44) degrees centigrade through the urethral tube in the prostate, e.g., to prevent a fatal temperature from being reached in its wall. An esophageal thermocouple also may be used to monitor the patient's body temperature and detect hypothermia.

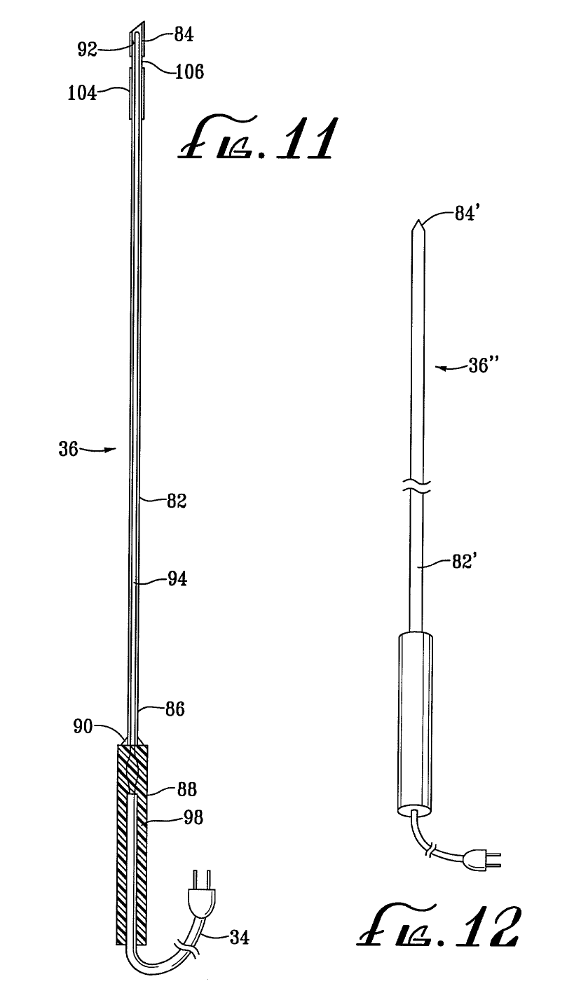

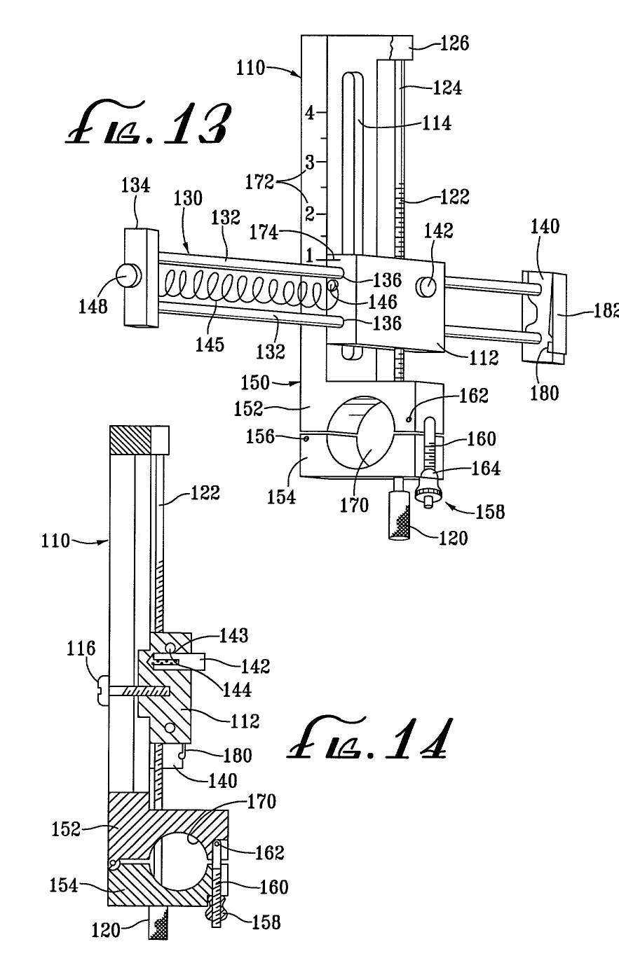

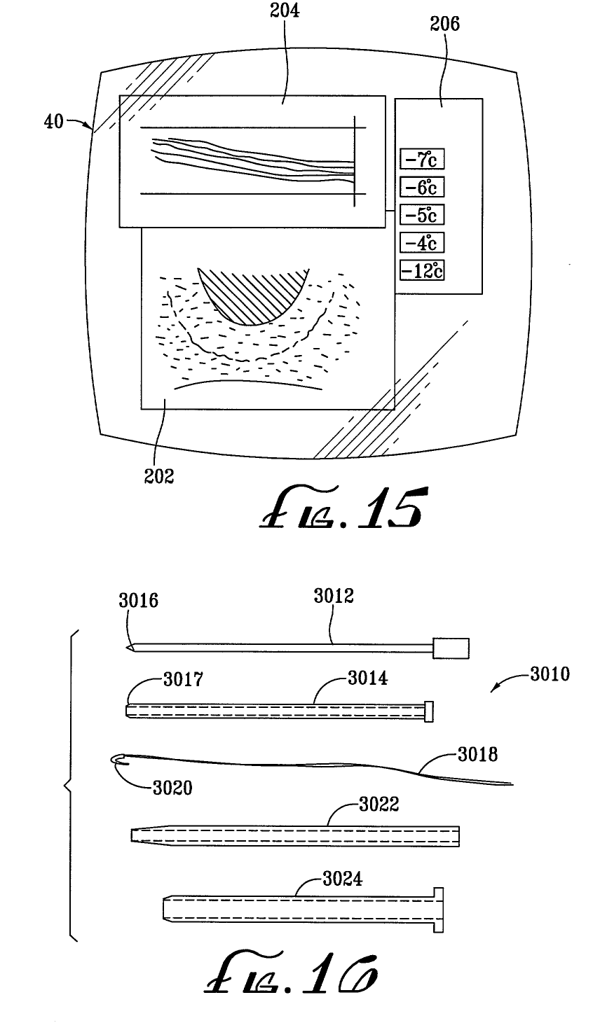

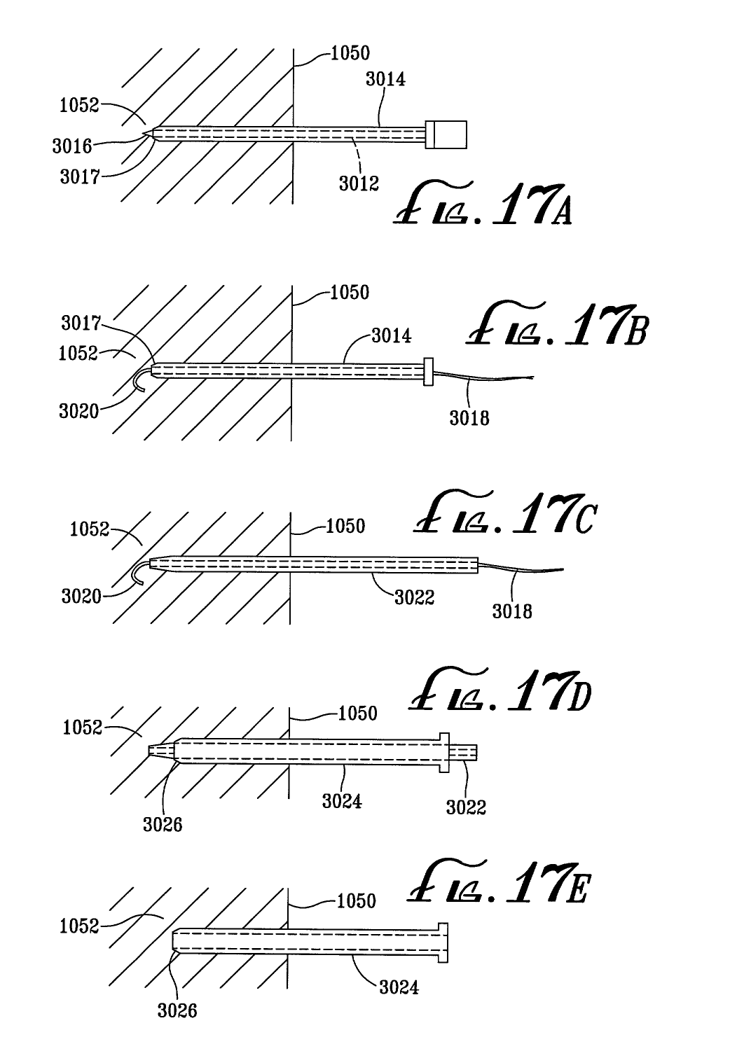

To place the various probes, particularly the cryogenic probes, a stereotactic guide, such as that shown in FIGS. 13 and 14, may be used. The guide generally includes a frame 110 on which a frame block 112 is mounted. More particularly, the frame 110 incorporates a slot 114 that receives the shaft of a screw 116 (shown in FIG. 14 and preferably of plastic) the shaft of which is threaded into frame block 112. The head of screw 116 may slide along the back surface of frame 110. Such movement conveniently may be effected using knurled knob 120 attached to a threaded shaft 122 that extends through a boss on frame block 112. The end 124 of the shaft may be received and captured in an opening in boss 126 projecting from frame 110. Thus, by rotating knob 120, it is possible, through the threaded engagement of shaft 122 with frame block 112, to cause the frame block to slide up and down along frame 110.

A guide bar 130, including rods 132 held in a parallel spaced relationship by a proximal block 134, may be received in and slide through openings 136 in frame block 112. The distal ends of rods 132 are captured in a guide block 140. A spring biased button 142 may be received in block 112 and may include a detent 143 that engages a notch 144 in rod 132 to hold the guide bar in a retracted orientation, as generally shown in FIG. 14. A spring 145 may be connected to the frame block by clamp 146 and to the proximal block 134 by clamp 148. The spring 145 tends to pull proximal block 134 toward frame block 112, but this motion is prevented while button 142 engages the notch in rod 132.

A positioning block may be provided at the base portion of frame 110 that includes an upper shell 152 and a lower shell 154 that are pivotally connected to one another by pivot pin 156. The shells 152, 154 may be clamped to one another by lock nut 158 received on threaded shaft 160 that ,in turn, is pivotally attached to upper shell 152 by pivot pin 162,. The threaded shaft 160 may be received in a recess in both upper shell 152 and lower shell 154, as shown. Preferably, the enlarged base 164 of lock nut 158 is received in an appropriately formed recess that encompasses more than a half circle of base 164, thereby to capture lock nut 158 and prevent it from moving out of this recess until it is turned down on threaded shaft 160 a distance to clear the bottom surface of lower shell 154. By this arrangement, the attachments of the shells to one another may be loosened to enlarge cylindrical opening 170 in shells 152 and 154 without releasing shell 154.

Opening 170 is slightly smaller than the outer diameter of the ultrasonic probe 16. Accordingly, by loosening lock nut 158, the size of opening 170 may be enlarged sufficiently to permit the ultrasonic probe to be passed through opening 170 and into a portion of the body to be illuminated by the probe, such as through anus 70. When the ultrasonic probe has been appropriately positioned, lock nut 158 may be turned down to clamp the upper and lower shells about the probe and to fix frame 110 relative to the probe. A series of graduations 172 may be provided on the back surface of the frame to permit the location of frame block 112 to be accurately or reproducibly located and positioned on frame 110 using marking 174 provided on the back surface of the frame block. This positioning may be effected by turning knurled knob 120 to cause its threaded shaft to drive frame block 112 up and down.

Guide block 140 may include a cylindrical opening 180 in its face that is closed by a sheet metal slide 182 shaped to envelope the guide block 140 and to be movable up and down along the guide block 140 to expose or close cylindrical opening 180 as desired.

By this configuration of the stereotactic guide, the various cryogenic probes and temperature probes that are desired to be used during the cryosurgical operation may each be accurately placed percutaneously within a body. Specifically, and again with reference to the prostate gland treatment generally illustrated in FIGS. 8 and 9, the guide may be clamped around ultrasonic probe 16 and located to orient cylindrical opening 180 at various predetermined distances spaced from the ultrasonic probe. The probes may then be passed through cylindrical opening 180, this opening guiding the shaft 82 of the temperature probe 36 along a path parallel to the ultrasonic probe to permit its accurate percutaneous placement within the body.

This placement may be selected and monitored using the cryogenic system shown in FIG. 7. Specifically, in one approach, the ultrasonic probe may be placed within the rectum and used to illuminate the prostate gland. To separate the rectal wall from the prostate gland, the tip of the ultrasonic probe may be moved and depressed against the posterior wall of the rectum. This may tend to pull the rectum off of the prostate (this technique may also be used during freezing). Preferably, each probe is inserted using the apparatus and method described above, until, a plurality of, e.g., five, cryogenic probes are passed through the sheaths into the prostate. After all the sheaths are in place, needles with trocars may be placed, under ultrasonic monitoring and/or freehand, at the (1) right neurovascular bundle, (2) left neurovascular bundle, and (3) at the 6:00 o'clock position between the rectum and posterior prostate. The longer 0.035 thermocouples may then be passed through the 18 gauge needles and into the desired depth, again using ultrasonic guidance. Two of the shorter thermocouples may be placed freehand without the use of the 18 gauge needles, but with the use of ultrasound (4) at the apex and (5) anterior edge of the prostate.

After placement of the probes, the cryogenic system 2 may be coupled to the probes and activated to gradually cause an ice ball to form and completely envelope the prostate gland. As this ice ball is forming, its nature and shape may be displayed on monitor 40, as generally indicated in the image or visual field presented in FIG. 15. Specifically, in the preferred arrangement, an ultrasonic image window 202 is provided that shows, as a blackened area, the gradual growth of the ice ball. Window area 204 preferably also is provided, this window area presenting the equivalent of a strip chart display of the recent temperatures detected by the various temperature probes 36 connected to acquisition system 32. Also, preferably in a window area such as at 206, various current temperature readings and other information may be presented to the user of the system. Thus, the various controls of cryogenic control system 6 may be manipulated to cause the ice ball to gradually grow and to sculpt the fatal temperature region to the shape of the gland, thereby to cryogenically kill the prostate gland. If desired, control system 6 also may be connected to data processor 30 to provide information about the degree of cooling being applied to and by the various cryogenic probes, and this information may be used to predict placement of the cryogenic probes and the extent of cooling (or zone of fatal temperature) that may result.

If the prostate is significantly enlarged, it may be necessary first to cause the ice ball to form in an area nearest bladder 64, then to move the cryogenic and temperature probes back somewhat to cause the ice ball to extend to the apex of the gland. Once the prostate gland or other defined internal region has been cooled to a fatal temperature, the cryosurgical system may be shut down. After the ice ball has stopped growing, the cooling probes may be removed and the temperature monitored until the ice ball gradually begins to dissipate. Then, the temperature probes may be removed together with the ultrasonic probe to conclude the operation. Thus, the system and procedure may achieve radical cryosurgical ablation of the target area and give positive assurance that a fatal temperature has been attained at the outer portions of the target area. It also may permit the freezing zone to be sculpted for the desired pattern but to be terminated before freezing occurs beyond the target area.

This cryosurgical procedure may be an effective modality for prostate carcinomas, even carcinoma modules extending somewhat beyond the prostate but connected to the prostate by the patient's circulatory system. Thus, this procedure may replace radical prostectomy and other similar procedures. The cryosurgical system and method, together with insertion apparatus, probes, and stereotactic guide, described herein, may provide a convenient and effective way to achieve such procedures.

Various changes and modifications in the preferred system and components may also be envisioned. For example, the temperature and/or thermocouple probes may be curved to better treat better various organs, such as tonsils and liver tissue. The temperature thermocouples may use “RTD” thermistors, which may be more reliable and accurate than thermocouples. The software and procedures for use may be modified to ensure accurate calibration of the thermocouples and data acquisition system. The cryogenic cooling system, and particularly its control system, may generate data that is supplied to the integrated data processor for display on monitor 40 and possibly even for prediction of the ice ball resulting from a particular application and cooling.

Many modifications to the apparatus 1005 discussed with respect to FIG. 1 may be implemented and still be within the scope of the present invention. For example, after radiation of an organ such as the prostate, fibroids may be formed that are difficult to pierce and dilate. In such an instance, a single dilation step may be difficult to perform. Alternatively, a relatively large cross-section introducer sheath 1030 may be needed, thus requiring a relatively large amount of dilation in the insertion path. The apparatus 1005 may be modified to perform multiple steps of dilation as may be necessary.

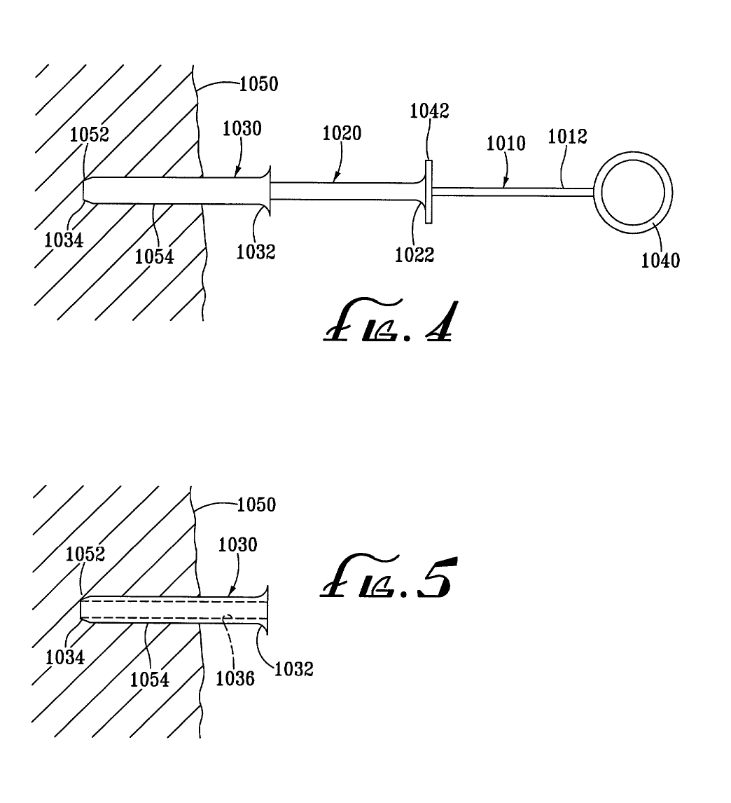

For example, turning to FIG. 6, an apparatus 2005 is shown that includes three nested dilators 2060, 2070, and 2080. Thus, instead of a single dilator, the apparatus 2005 allows three successive steps of dilation to ease the overall dilation task. The inner dilator 2060 generally contains a stylet 2010 in its lumen 2066. In turn, the middle dilator 2070 may contain inner dilator 2060 in its lumen 2076. A tapered distal end 2074 of middle dilator 2070 may taper substantially from an outer diameter of the inner dilator 2060 to an inner diameter of the outer dilator 2080. In turn, the outer dilator 2080 may contain middle dilator 2070 in its lumen 2086 such that a tapered distal end 2084 of the outer dilator 2080 may taper substantially from the outer diameter of the middle dilator 2070 to a final outer diameter required for outer dilator 2080, e.g., to accommodate advancement of an introducer sheath (not shown).

Proximal ends (not shown) of the nested dilators 2060, 2070, 2080 may include a series of luer locks or other mechanisms (also not shown), similar to that described above with respect to FIGS. 1A and 1B. Thus, inner dilator 2060 may have a male luer coupler on its outer surface at its proximal end, and the middle dilator 2070 may have a female luer coupler within its lumen at its proximal end such that inner dilator 2060 and middle dilator 2070 may be connected together. Similarly, the middle dilator 2070 may have a male luer coupler on its outer surface at its proximal end, and the outer dilator 2080 may have a female luer coupler within its lumen at its proximal end such that the middle dilator 2070 and the outer dilator 2080 may be connected together. Finally, similar to the embodiment described above with respect to FIGS. 1A and 1B, the outer dilator 2080 may have a female luer coupler within its lumen at its proximal end to connect with a male luer coupler at the proximal end of the stylet 2010. In addition, outer dilator 2080 (or each of the dilators) may have a flange (not shown), similar to the flange of FIGS. 1A and 1B, or a pistol grip, as described above, and/or the stylet 2010 may include a thumb ring (not shown).

It will be appreciated that any number of dilators may be used, as needed by a particular amount of dilation. The method of using this apparatus 2005 having nested dilators is analogous to the method described above with respect to FIGS. 2-5. Initially, after insertion of the stylet 2010, the innermost dilator 2060 may be disconnected (if interlocking mechanisms are employed) from the other dilators. The innermost dilator 2060 may then be advanced distally along the stylet 2010 to perform the first stage of the dilation. Then, the dilator 2070 adjacent the innermost dilator 2060 may be disconnected and advanced and so on until the outermost dilator 2080 has been disconnected from the stylet 2010 and advanced. At this point, the introducer sheath (not shown) may be advanced and located with the dilated insertion path.

Specific examples of the present invention have been shown by way of example in the drawings and are herein described in detail. It is to be understood, however, that the invention is not to be limited to the particular forms or methods disclosed, but to the contrary, the invention is to broadly cover all modifications, equivalents, and alternatives encompassed by the scope of the appended claims.

What is claimed is:

1. A system for performing a cryogenic procedure at an internal tissue region, comprising:
a stylet having a proximal end and a distal end for penetrating tissue;
a dilator slidably disposed on the stylet, the dilator having a proximal end and a tapered distal end for substantially atraumatically dilating tissue, the dilator having a length that is less than a length of the stylet;
a lock assembly on the proximal ends of the stylet and the dilator for securing the dilator axially with respect to the stylet such that a distal portion of the stylet extends beyond the distal end of the dilator;
an introducer sheath slidably disposed on the dilator; the dilator being movable proximally with respect to the introducer sheath for removing the dilator from within the introducer sheath; and
a cryogenic probe insertable into the introducer sheath when the dilator and stylet are removed from within the introducer sheath.

2. The system of claim 1, wherein the lock assembly is configured for detachably securing the proximal ends of the stylet and the dilator with respect to one another.

3. The percutaneous access system of claim 2, wherein the lock assembly comprises a luer lock.

4. The percutaneous access system of claim 1, wherein the stylet has a thumb ring on its proximal end for facilitating advancement of the stylet into tissue.

5. The system of claim 1, wherein a distal end of the introducer sheath is tapered.

6. The system of claim 1, comprising a plurality of cryogenic probes, each individually insertable into the introducer sheath.

7. The system of claim 1, further comprising a cooling system coupled to the probe for cooling a distal portion of the cryogenic probe sufficient to ablate tissue with the distal portion.

8. The system of claim 7, further comprising an ultrasound imaging system for monitoring placement of at least one of the stylet, the dilator, the introducer sheath, and the cryogenic probe.

9. The system of claim 7, further comprising one or more temperature probes, the temperature probes configured for introduction into the internal tissue region.

10. The system of claim 9, further comprising a processor for monitoring temperature data using the one or more temperature probes and adjusting the cooling system in response to the temperature data to control the cooling of the distal portion of the cryogenic probe.

11. An apparatus for providing percutaneous access for a medical procedure, comprising:
a stylet having a proximal end and a distal end for penetrating tissue;
a dilator slidably disposed on the stylet, the dilator having a proximal end and a tapered distal end, the dilator having a length that is less than a length of the stylet;
an introducer sheath slidably disposed on the dilator; and
a mechanism for limiting proximal movement of the dilator with respect to the stylet to prevent the proximal end of the stylet from being advanced distally through the dilator.

12. The apparatus of claim 11, wherein the mechanism comprises a lock assembly on the proximal ends of the stylet and the dilator for axially securing the dilator with respect to the stylet such that a distal portion of the stylet extends beyond the distal end of the dilator.

13. The apparatus of claim 12, wherein the lock assembly is configured for detachably securing the proximal ends of the stylet and the dilator with respect to one another.

14. The apparatus of claim 12, wherein the lock assembly comprises a luer lock.

15. The apparatus of claim 1 1, wherein the stylet is removable proximally from within the dilator.

16. The apparatus of claim 11, wherein the dilator is movable proximally with respect to the introducer sheath for removing the dilator from within the introducer sheath.

17. The apparatus of claim 16, further comprising a cryogenic probe insertable into the introducer sheath once the dilator is removed from within the introducer sheath.

18. The apparatus of claim 11, wherein the mechanism comprises a thumb ring on the proximal end of the stylet.

19. The apparatus of claim 18, wherein the mechanism further comprises a flange on the proximal end of the dilator for preventing the proximal end of the dilator from being advanced into the introducer sheath.

20. The apparatus of claim 1 1, wherein a distal end of the introducer sheath is tapered.

21. A method for performing a cryogenic procedure, comprising:
providing an apparatus comprising a stylet, a dilator secured about a proximal portion of the stylet such that a distal portion of the stylet extends beyond a tapered distal end of the dilator, and an introducer sheath disposed on the dilator;
inserting the distal portion of the stylet through a patient's skin to a target tissue region;
advancing the dilator such that the tapered distal end of the dilator substantially atraumatically dilates tissue surrounding the distal portion of the stylet until the distal end of the dilator is coextensive with a distal end of the stylet, thereby creating a dilated insertion path;
advancing the introducer sheath until a distal end of the introducer sheath is aligned with a distal end of the dilated insertion path; and
removing the dilator and stylet, leaving the introducer sheath in the dilated insertion path.

22. The method of claim 21, further comprising removing the stylet and the dilator from the introducer sheath after the distal end of the introducer sheath is aligned with the distal end of the dilated insertion path.

23. The method of claim 21, further comprising inserting a cryogenic probe into the introducer sheath after the step of removing the stylet and the dilator, the cryogenic probe being inserted until a distal end of the probe is aligned with or extends beyond the distal end of the dilated insertion path.

24. The method of claim 23, further comprising cooling the distal end of the probe until a target tissue region surrounding the distal end of the probe is substantially ablated.

25. The method of claim 24, further comprising measuring a temperature of the target tissue region as the distal end of the probe is cooled.

26. The method of claim 25, wherein the step of measuring a temperature comprises introducing one or more temperature probes into the patient in a predetermined relationship with the target tissue region, and wherein the one or more temperature probes measure the temperature of the target tissue region.

27. The method of claim 24, further comprising imaging the target tissue region to monitor an extent of ablation.

28. The method of claim 27, wherein the target tissue region is imaged using ultrasonic imaging.

29. The method of claim 21, wherein the insertion of the stylet is performed through a percutaneous incision.

30. The method of claim 21, wherein the stylet is inserted directly into the patient's skin.
